(19) United States
(12) Patent Application Publication
(10) Pub. No.: US 2003/0124024 A1
(43) Pub. Date: Jul. 3, 2003

(54) Dry-type dehydrating and sanitizing device

(76) Inventor: Yuan-Fu Chang, Feng-Yuan City (TW)

Correspondence Address: CHARLES E. BAXLEY, ESQUIRE, 59 JOHN STREET 5TH FL., NEW YORK, NY, 10038, US

(21) Appl. No.: 10/035,270

(22) Filed: Jan. 3, 2002

(51) Int. Cl. A61L 2/08

(52) U.S. Cl. 422/22; 422/297; 422/299; 422/302

(57) ABSTRACT

A dehydrating and sanitizing device for baby stuff includes a bowl with a heating member received therein and an inclined plate is connected to the heating member so as to guide water to flow in a collection drawer located at a lower edge of the inclined plate. Several sanitizing bulbs are connected to the inside of the bowl. A rack is located in the bowl for the nursing bottles and teats being put thereon and a cover is mounted to an open top of the bowl.

### FIELD OF THE INVENTION

The present invention relates to a dehydrating and sanitizing device for sanitizing nursing bottles and teats which are dry after being treated by the device.

### BACKGROUND OF THE INVENTION

A conventional sanitizing device for sanitizing baby stuff such as the nursing bottles is shown in FIG. 1 and generally includes a bowl 10 and a cover 12 which is mounted on the open top of the bowl 10. A heating member 13 is located in the bowl 10 and a plate 14 is connected on the heating member 13. A rack 12 is located above the plate 14 so that the nursing bottles 15 can be put on the rack 12. Water is put in the plate 14 and heated by the heating member 13 so as to generate vapor which sanitizes germs in the nursing bottles 15. After the vapor is cooled, it drained into the plate 14 again. However, water drops are attached on the nursing bottles 15 when the users want to use them and therefore the users have to wipe the water drops before using the nursing bottles 15. The clothes or tissues used to wipe the nursing bottles 15 involve chemical products such as blend agent or fluorescent agent which is not good for infants. Furthermore, the water collected in the plate 14 from cooled vapor becomes a germ-rich spot if the plate 14 is not frequently cleaned.

### SUMMARY OF THE INVENTION

In accordance with one aspect of the present invention, there is provided a dehydrating and sanitizing device which comprises a bowl having a heating member received therein and an inclined plate is connected to the heating member. A collection drawer is removably engaged with the bowl and located at a lower edge of the inclined plate. At least one sanitizing bulb is connected to inside of the bowl and a rack is located in the bowl and above the at least one sanitizing bulb and the heating member. A cover is mounted to an open top of the bowl.

The primary object of the present invention is to provide a dehydrating and sanitizing device that uses sanitizing bulbs and heating member to sanitize the nursing bottles and teats without leaving water drops on the nursing bottles and teats.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a cross sectional view to show a conventional sanitizing device for baby stuff;

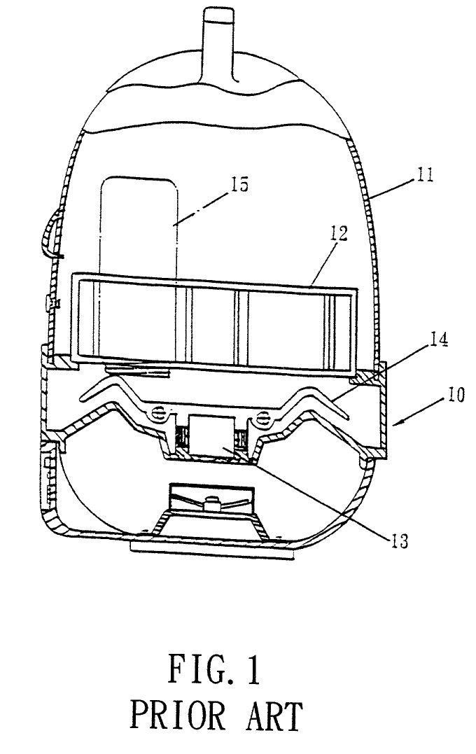

FIG. 2 is an exploded view to show a dehydrating and sanitizing device of the present invention, and

FIG. 3 is a perspective view to show the dehydrating and sanitizing device of the present invention.

### DETAILED DESCRIPTION OF THE PREFERRED EMBODIMENT

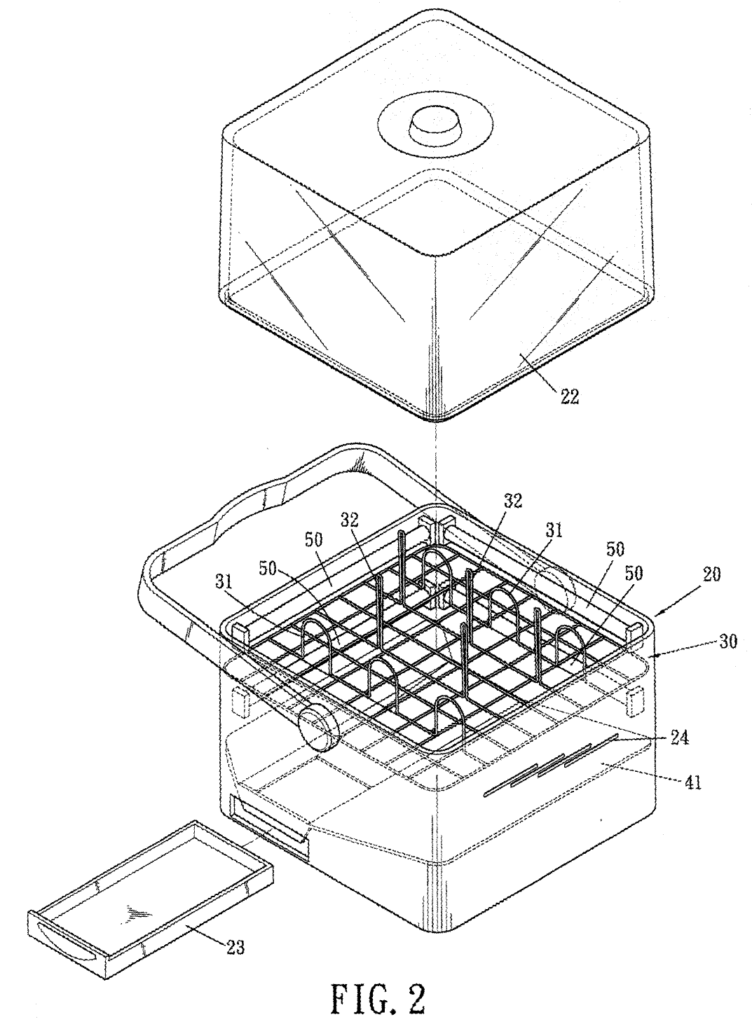

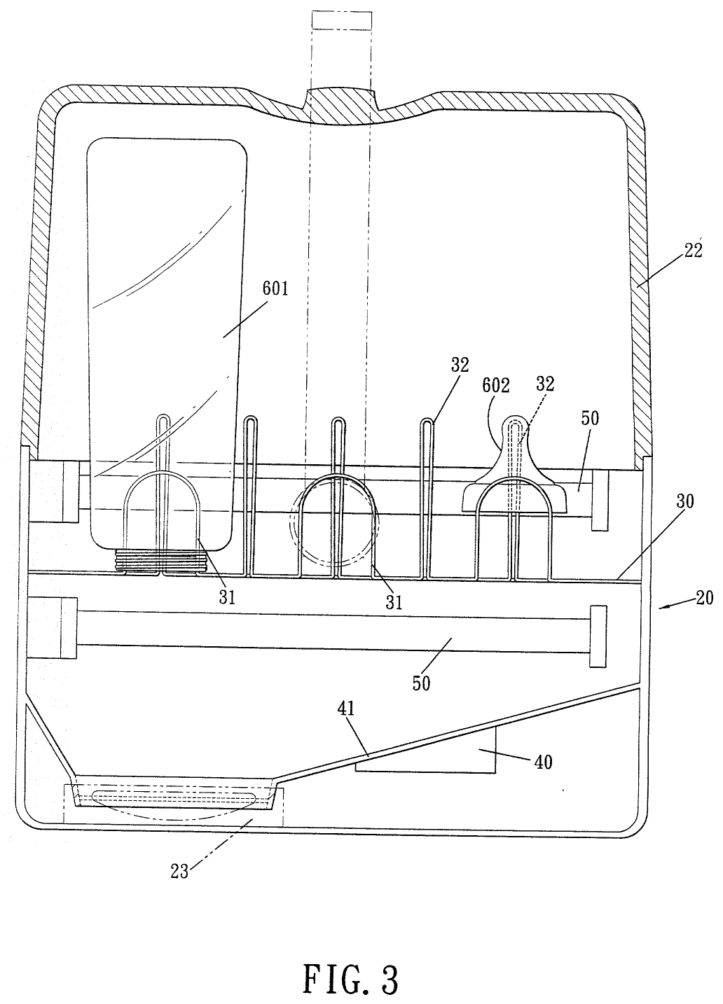

Referring to FIGS. 2 and 3, the dehydrating and sanitizing device of the present invention comprises a bowl 20 having a heating member 40 received therein and an inclined plate 41 is connected to the heating member 40. A collection drawer 23 is removably engaged with an opening on a side of the bowl 20 the collection drawer 23 is located at a lower edge of the inclined plate 41. A plurality of sanitizing bulbs 50 are connected to the inside of the bowl 20. A plurality of ventilation apertures 24 are defined through walls of the bowl 20.

A rack 30 is located in the bowl 20 and a plurality of protrusions 31, 32 extend from the rack 30. The rack 30 is located above the sanitizing bulbs 50 and the heating member 40 so that the nursing bottles 601 and teats 602 are put on the protrusions 31, 32. A cover 22 is mounted to an open top of the bowl 20.

When using the device, the heating member 40 is activated to increase the temperature in the device so as to sanitize the bottles 601 and the teats 602 by transferring the water drops attached on the bottles 601 and the teats 602 into vapor. The sanitizing bulbs 50 kill the germs and are maintained in operation status to ensure no bottles 601 and teats 602 are to be contaminated. The collection drawer 23 collects water cooled from vapor in the device and the water flows in the collection drawer 23 along the inclined plate 41. The device needs no water to be added and can dehydrate the bottles 601 and the teats 602.

While we have shown and described the embodiment in accordance with the present invention, it should be clear to those skilled in the art that further embodiments may be made without departing from the scope of the present invention.

What is claimed is:

1. A dehydrating and sanitizing device comprising:
a bowl having a heating member received therein and an inclined plate connected to said heating member, a collection drawer removably engaged with said bowl and located at a lower edge of said inclined plate, at least one sanitizing bulb connected to an inside of said bowl, a plurality of ventilation apertures defined through walls of said bowl;
a rack located in said bowl and above said at least one sanitizing bulb and said heating member, and
a cover mounted to an open top of said bowl.

2. The device as claimed in claim 1 further comprising a plurality of protrusions extending from said rack.
